(19) United States
(12) Patent Application Publication
(10) Pub. No.: US 2021/0346141 A1
(43) Pub. Date: Nov. 11, 2021

(54) Surgical device for correction of upper eyelid ptosis with fascial or tendon sling

(71) Applicants: Afzaal Bashir Bajwa, Pasrur (PK); Sunaina Afzaal, Pasrur (PK); Sadia Rehman Shaikh, Pelham, AL (US)

(72) Inventors: Afzaal Bashir Bajwa, Pasrur (PK); Sunaina Afzaal, Pasrur (PK); Sadia Rehman Shaikh, Pelham, AL (US)

(21) Appl. No.: 17/214,941

(22) Filed: Mar. 29, 2021

(30) Foreign Application Priority Data
May 10, 2020 (PK) 678/2020

(51) Int. Cl. A61F 2/00

(52) U.S. Cl. CPC A61F 2/0063; A61F 2002/0072; A61F 2/0059

(57) ABSTRACT

A surgical device for correction of upper eyelid ptosis with autologous sling, comprising: a malleable rod (1), 6 cm long and 3 mm thick with blunt tapering tip (2); a catching end (3), where the catching end (3) contains both upper jaw (3A), lower jaw (38), where plurality of forward directed teeth (3C) positioned on both jaws inner surface; a hinge (3D) with closing spring, keeping both the jaws closed; an oval central hole (3E) of 2 mm positioned in mid of both the jaws so the upper hole faced towards the lower hole.

The device holds the sling and passes from one point of eyelid to the other in 5 seconds to 10 seconds.

### FIELD OF INVENTION

Present invention relates to the field of medical instruments in particular to a field of eye lid surgery, specifically, a device for passing fascial or tendon sling in upper eyelid ptosis correction.

### BACKGROUND OF INVENTION

Upper eyelid ptosis may be congenital or acquired and results in inability to open upper eyelid. Servat, J. J., & Baylin, E. B. (2018). Surgical Anatomy of the Eyelid Oculoplastic Surgery Atlas (pp. 1-12): Springer. To treat such deformity, surgeon has to insert synthetic or autologous sling. Rosenberg. J. B., Andersen, J., & Barmettler, A. (2019). Types of materials for frontalis sling surgery for congenital ptosis. Cochrane Database of Systematic Reviews (4). This sling empowers frontalis muscle to elevate upper eyelid. Synthetic stings are easily available in the market but are associated with infection, extrusion and cost issues. In contrast, autologous sling are made by harvesting fascia or tendons and are more safe and convenient. Nucci, P., Lembo. A., Santangelo, E., Fogagnolo. P., & Serafino. M. (2016). Five-year follow-up of a 30-month trial of stability of silicone band frontalis suspension for the treatment of severe unilateral upper eyelid ptosis in infants. Paper presented at the Seminars in Ophthalmology.

Placement of autologous sling requires tunnel formation through which sling is passed. Traditionally, tunnel is formed with the help of forceps or scissor tips. Being not only laborious, traditional methods require multiple passes of instruments and lead to lot of postoperative swelling with prolonged healing. Moreover, tunnelling and tendon passing is done one after the other. Hamama, J., Khalfi. L., Sabani. H., & El Khatib, K. (2018). Frontalis suspension using autogenous temporal fascia by Fox technique. Journal of stomatology, oral and maxillofacial surgery, 119(4), 311-314.

Sling passing with this novel device not only makes the tunnelling easy but also passes the tendon simultaneous in a single go, saving time and helping in quicker recovery postoperatively.

### DESCRIPTION OF INVENTION

Unlike previous synthetic slings known and described in prior art, the sling of present invention is a part of patient's own natural fascia or tendon which is sliced into thin long strap like pieces of 2-5 mm wide. The said slings are placed in upper eyelid to forehead by making tunnels with the help of surgical device, “Ptosis Sling Passer” which is the subject matter of present invention.

The surgical device of present invention serves purpose of creation of tunnel for passing sling simultaneously. This device holds the sling with the help of forward directed teeth on the inner side and helped by spring closure.

Unless defined otherwise all technical and scientific terms used herein have the same meanings as commonly understood to one of ordinary skill in the art to which this invention belongs.

The details of one or more embodiments of the invention are set forth in the accompanying drawings and the description below. Other features, objects and advantages of the invention will be apparent from the description, drawings and from the claims.

Objects of the present invention include providing a surgical device with its components and a method of using the device. Claimed device of present invention is an innovative approach in Upper Eyelid Ptosis correction using fascial or tendon sling and passing the sling from one point to the other in 5 to 10 seconds with less trauma. Further, as post-operative activity there is less oedema and quick recovery.

A surgical device for Upper Eyelid Ptosis correction, comprising: a body (1), where the body (1) is 6 cm long and 3 mm thick metallic rod, blunt tapering end (2) is the leading end and tapers over 5 mm, a Catching end (3) which has upper jaw (3A), lower jaw (3B), multiple forward directed teeth (3C) on both jaws on inner side, a hinge with closing spring (3D) joining both jaws and keeping them closed and oval central hole (3E) of 2 mm in mid of both jaws.

The tapering end helps in making a tunnel in the soft tissues of upper eyelid and catching end holds the sling while passing through the tunnels from one point to the other. The malleable body makes it easy to pass sling through layers of upper eyelid to forehead of patient at different points.

A surgical device for use in upper eyelid ptosis correction comprises of following steps:
Holding the sling of fascia or tendon in the jaws of catching end (3) with the help of teeth and spring;
pushing the tapering end (2) in the layers of upper eyelid at marked points while putting the pressure of fingers on the skin;
advancing the device;
moving out the surgical device gradually; and
releasing the sling by opening the jaws

A method of using a surgical device for correction of upper eyelid ptosis with sling comprising number of steps, before using said device, a sling is taken or cut from patient's own fascia or tendon. Said sling is cut or sliced with surgical knife in surgical tray. The sling is held in catching end and passed by creating tunnel in the eyelid tissue with the tapering end. Device is slowly passed to the other side of the tunnel and jaws are opened to unload the sling.

### DESCRIPTION OF DRAWINGS

Description of drawings is as follows:

FIG. 1: FIG. 1 shows the lateral longitudinal view of claimed device. FIG. 1 mentions all parts of device like Body (1) blunt tapering end (2) and catching end (3). Body (1) is 6 cm long metallic rod which is 3 mm thick. The tapering end is blunt and tapers 3 mm long. Catching end (3) has upper jaw (3A), lower jaw (3B), forward directed teeth (3C) on both jaws, spring containing hinge (3D) attaching two jaws which keeps jaws closed.

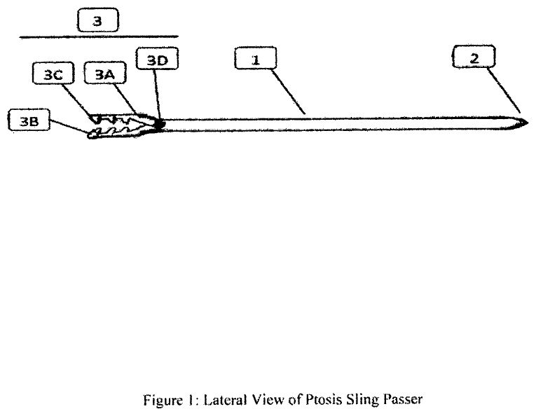

FIG. 2: FIG. 2 is dorsal view showing rod like body (1) along with tapering end (2) and catching end (3) with central 2 mm hole (3E).

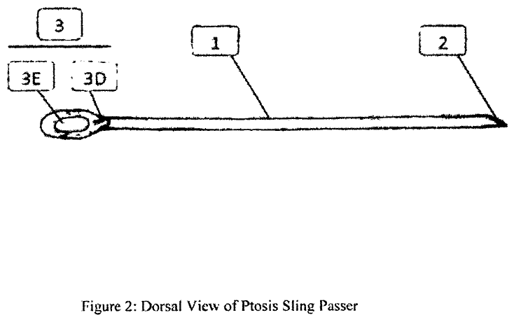

We claim:

1. A surgical device for correction of upper eyelid ptosis with autologous sling, comprising: a malleable rod (1), 6 cm long and 3 mm thick with blunt tapering end (2); a catching end (3), where the catching end (3) contains both upper jaw (3A), lower jaw (3B), where plurality of forward directed teeth (3C) positioned on both jaws inner surface; a hinge (3D) with closing spring, keeping both the jaws closed to hold the sling; an oval central hole (3E) of 2 mm positioned in mid of both the jaws so the upper hole faced towards the lower hole to safe the tendon from crushing.

2. A surgical device as claimed in claim 1, where the Body is malleable rod easily bent around 360° angle.

3. A surgical device as claimed in claim 1, where a malleable rod (1), is 6 cm long 3 mm thick.

4. A surgical device as claimed in claim 1, where tapering end (2) is blunt and tappers over 5 mm.

5. A surgical device as claimed in claim 1, where catching end (3) is lcm long and 5 mm wide and has upper jaw (3A) and lower jaw (3B).

6. A surgical device as claimed in claim 1, where catching end (3) has hinge (3D) with closed spring to hold the sling.

7. A surgical device as claimed in claim 1, where catching end has a hinge (3D) attaching jaws (3A) and (3B).

8. A surgical device as claimed in claim 1, where the attaching end has a central hole (3E) which is 2 mm wide.

9. A surgical device as claimed in claim 1, where an oval central hole (3E) of 2 mm helps in through and through placement of the stitch to hold the sling tightly.

10. A surgical device as claimed in claim 1, holds the sling and passes from one point of eyelid to the other in 5 seconds to 10 seconds.

11. A surgical device as claimed in claim 1, for the use in upper eyelid ptosis correction comprises following steps:
holding the sling of fascia or tendon in the jaws of catching end (3) with the help of teeth and spring;
pushing the tapering end (2) in the layers of upper eyelid at marked points while putting the pressure of fingers on the skin;
advancing the device;
moving out the surgical device gradually; and
releasing the sling by opening the jaws.
